(19) United States
(12) Patent Application Publication
(10) Pub. No.: US 2013/0123903 A1
(43) Pub. Date: May 16, 2013

(54) ENHANCED RETENTION STENT

(71) Applicant: Diem U. Ta, San Jose, CA (US)

(72) Inventor: Diem U. Ta, San Jose, CA (US)

(73) Assignee: ABBOTT CARDIOVASCULAR SYSTEMS, INC., Santa Clara, CA (US)

(21) Appl. No.: 13/295,583

(22) Filed: Nov. 14, 2011

(51) Int. Cl. A61F 2/82

(52) U.S. Cl. 623/1.16

(57) ABSTRACT

An expandable stent has larger cells located at the proximal and distal ends of the stent than in the body portion so that more of a catheter balloon can protrude into the stent cells to increase stent retention. The intravascular stent has a plurality of cylindrical rings connected by links, the spacing of which is a factor in defining the cell size. Where the spacing between adjacent rings is longer such as at the ends of the stent, more than one U-shaped elements can be incorporated into the links to improve flexibility. The stent can be compressed or crimped onto a balloon catheter to a very low profile and maintain a high degree of stent retention due to increased spacing between rings at the proximal and distal ends of the stent.

### BACKGROUND

This invention relates to vascular repair devices, in particular intravascular stents, which are to be implanted into a patient's body lumen, such as an artery or a coronary artery or a bile duct to maintain patency thereof. It is an important feature of the present invention to provide a stent structure that can be crimped onto a balloon catheter to form a high degree of stent retention so that during delivery of the stent to the treatment site in a coronary artery or other vessel or duct, the stent remains on the catheter.

Stents are generally tubular-shaped devices which function to hold open a segment of a blood vessel or other body lumen such as a renal or coronary artery. At present, there are numerous commercial stents being marketed throughout the world. While some of these stents are flexible and have the appropriate radial rigidity needed to hold open a vessel or an artery, there typically is a tradeoff between flexibility and radial strength and the ability to be tightly compressed or crimped onto a catheter balloon, so that it does not move relatively to the catheter or dislodge prematurely prior to controlled implantation in a vessel.

Currently, to secure a stent on a balloon, after the stent is crimped onto the balloon, the delivery system is pressurized such that the balloon would protrude through the stent struts. During this process, the balloon and stent are placed in a heated mold. This pressure and heat process retains the balloon protrusion through the stent struts. This balloon protrusion then acts as bumps to secure the stent in place.

Stent retention is a safety concern of all stent systems. Insufficient stent retention can lead to the loss of stents in-vivo resulting in a potentially dangerous condition for the patient. Therefore, there has been and remains a significant need for improved stent retention methods and devices. The prior art has sought to address the problem of stent retention in various ways. For example, various securement features such as bumps, ridges, and the like have been attempted to retain the stent on the balloon as it is delivered to the implant site. It is also known to overlay the stent with a sleeve or cover to prevent the stent from being dislodged as the catheter navigates the patient's vascular system. The success of many of these attempted solutions are questionable, particularly when the benefit to cost is examined. Therefore, a more efficient method of improving stent retention is still sought in the art.

In particular, what has been needed and heretofore unavailable is a stent pattern which has a high degree of flexibility and security, so that it can be advanced through tortuous vasculature while remain tightly crimped on a balloon catheter during delivery and yet have the mechanical strength to hold open a body lumen or an artery into which it is implanted and provide adequate vessel wall coverage. The stent pattern of the present invention satisfies this need.

### SUMMARY OF THE INVENTION

The stent pattern of the present invention provides larger spacing between the rings at the ends of the stent and their neighboring ring to allow more balloon material to protrude through the cells of the rings as the delivery system is pressurized after the stent has been crimped onto the balloon. Balloon protrusion acts as bumps to secure the stent in place. More balloon material captured between the rings at the ends of the stent enhances the stent's adherence to the balloon, and the goals of the present invention are served. The additional space between the rings at the ends of the stent and their neighboring ring further allows the adjoining links to have greater resiliency which enhances the flexibility of the stent.

In one embodiment of the stent of the present invention, each of the cylindrical rings making up the stent has a proximal end and a distal end and a cylindrical plane defined by a cylindrical outer wall surface that extends circumferentially between the proximal end and the distal end of the cylindrical ring. Generally, the cylindrical rings have a serpentine or undulating shape which includes at least one W-shaped element, and typically each ring has more than one W-shaped element. The cylindrical rings are interconnected by links which attach one cylindrical ring to an adjacent cylindrical ring. In this embodiment, all the rings are positioned in phase relatively to one another, and all of the connecting links are substantially straight and substantially parallel to the longitudinal axis of the stent. They connect one ring to the next by connecting the W crest in one ring to the Y crest in the adjacent ring. Each link has a U-shaped element that provides flexibility to the structure. In order to further improve stent retention on the expandable member (or balloon), the gaps between the rings at the ends of the stent and their neighboring ring are greater than the gaps between the rings in the main body of the stent. This feature increases stent retention on the catheter balloon since more balloon gets to protrude into the larger gaps at both ends of the stent to secure the stent on the balloon.

These and other features and advantages of the present invention will become apparent from the following detailed description of the preferred embodiments which, taken in conjunction with the accompanying drawings, illustrate by way of example the principles of the present invention.

### BRIEF DESCRIPTION OF THE DRAWINGS

FIG. 1 is an elevated view, partially in section, of an embodiment of a stent of the present invention mounted on a rapid-exchange delivery catheter and positioned within an artery;

FIG. 1a is an enlarged sectional view of the distal end of the stent crimped on the catheter balloon showing the protruding portion of the balloon through the stent struts;

FIG. 2 is an elevated view, partially in section, similar to that shown in FIG. 1 wherein the present invention is expanded within an artery so that the stent embeds within the arterial wall;

FIG. 3 is an elevated view, partially in section, showing the expanded stent after withdrawal of the rapid-exchange delivery catheter;

FIG. 4 is a plan view of a flattened stent of a prior art stent that illustrates the pattern of rings and links; and

FIG. 5 is a plan view of one embodiment of a flattened stent of the present invention illustrating the pattern of rings and links.

### DETAILED DESCRIPTION OF THE PREFERRED EMBODIMENTS

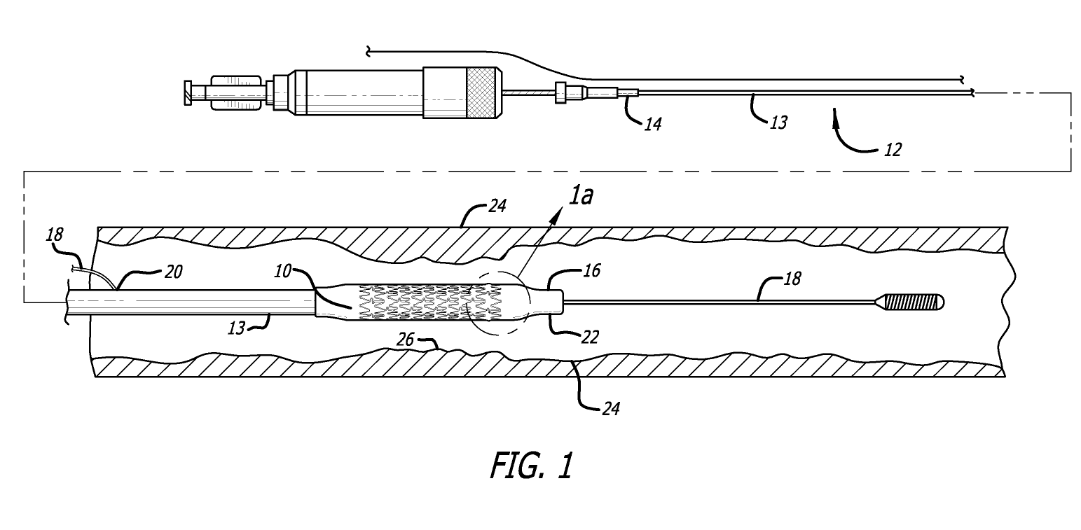

FIG. 1 depicts one environment of the present invention showing stent 10 mounted on a catheter assembly 12 which is used to deliver the stent and implant it in a body lumen, such as a coronary artery, peripheral artery, or other vessel or lumen within the body. The catheter assembly includes a catheter shaft 13 which has a proximal end 14 and a distal end 16. The catheter assembly is configured to advance through the patient's vascular system over a guide wire 18 by any of the well known methods of an over the wire system (not shown) or a well known rapid exchange catheter system, such as the one shown in FIG. 1.

Catheter assembly 12 as depicted in FIG. 1 is of the well known rapid exchange type which includes an RX port 20 where the guide wire 18 exits the catheter. The distal end of the guide wire 18 exits the catheter distal end 16 so that the catheter advances over the guide wire on a section of the catheter between the RX port 20 and the catheter distal end 16. As is known in the art, the guide wire lumen that receives the guide wire is sized for receiving various diameter guide wires to suit a particular application. The stent 10 is mounted on an expandable member 22 such as an inflatable balloon and is crimped tightly thereon so that the stent 10 and expandable member 22 present a low profile diameter for delivery through the arteries.

As shown in FIG. 1, a partial cross-section of an artery 24 is shown with a small amount of plaque 26 that has been previously treated by an angioplasty or other repair procedure. Stent 10 of the present invention may be used to repair a diseased arterial wall that has plaque 26 as shown in FIG. 1, a dissection, or a flap as is commonly found in the coronary arteries, peripheral arteries, and other vessels.

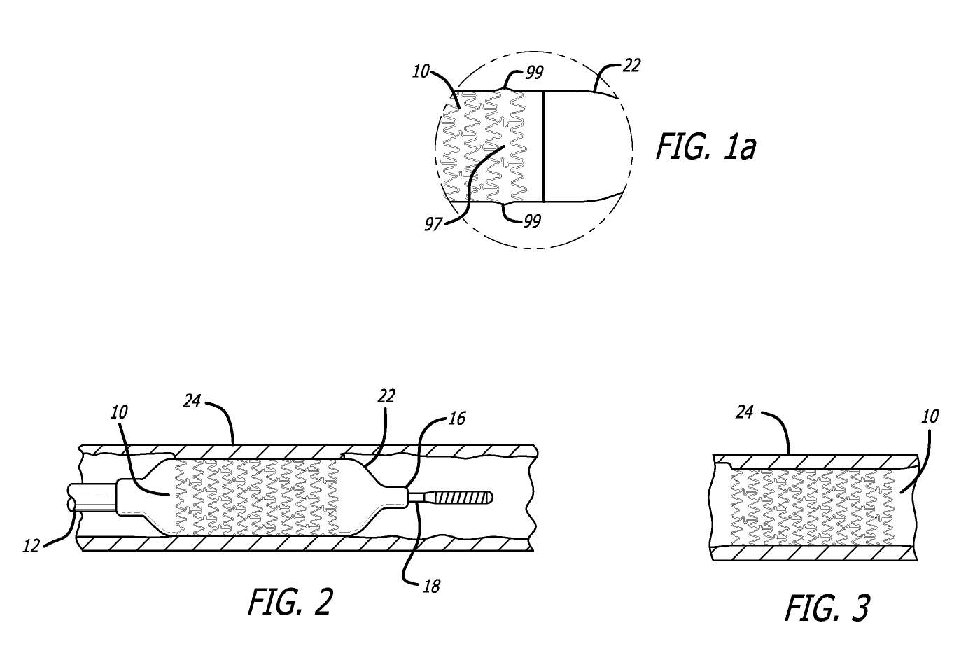

In a typical procedure to implant stent 10, the guide wire 18 is advanced through the patient's vascular system by well known methods so that the distal end of the guide wire is advanced past the plaque 26 or diseased area. Prior to implanting the stent 10, the cardiologist may wish to perform an angioplasty procedure or other procedure (i.e., atherectomy) in order to open the vessel and remodel the diseased area. Thereafter, the stent delivery catheter assembly 12 is advanced over the guide wire 18 so that the stent is positioned in the target area. The expandable member 22 is inflated by well known means so that it expands radially outwardly and in turn expands the stent 10 radially outwardly until the stent 10 bears against the vessel wall. The expandable member 22 is then deflated, and the catheter is withdrawn from the patient's vascular system, leaving the stent 10 in place to dilate the body lumen. The guide wire 18 typically is left in the lumen for post-dilatation procedures, if any, and subsequently is withdrawn from the patient's vascular system. As depicted in FIGS. 2 and 3, the inflatable member 22 is fully inflated with the stent 10 expanded and pressed against the vessel wall, and in FIG. 3, the implanted stent remains in the vessel after the balloon has been deflated, and the catheter assembly and guide wire have been withdrawn from the patient.

The stent 10 serves to hold open the artery after the catheter is withdrawn, as illustrated by FIG. 3. Due to the formation of the stent from an elongated tubular member in this particular embodiment, the undulating components of the stent are relatively thin in transverse cross-section, so that when the stent is expanded, it is pressed into the wall of the artery and, as a result, does not interfere significantly with the blood flow through the artery or damage the arterial wall. The stent is pressed into the wall of the artery and will eventually be covered with endothelial cell growth which further minimizes blood flow interference. The undulating portion of the stent provides good tacking characteristics to prevent stent movement within the artery. Furthermore, the closely spaced cylindrical rings at regular intervals provide uniform support for the wall of the artery and, consequently, are well adapted to tack up and hold in place small flaps or dissections in the wall of the artery, as illustrated in FIGS. 2 and 3.

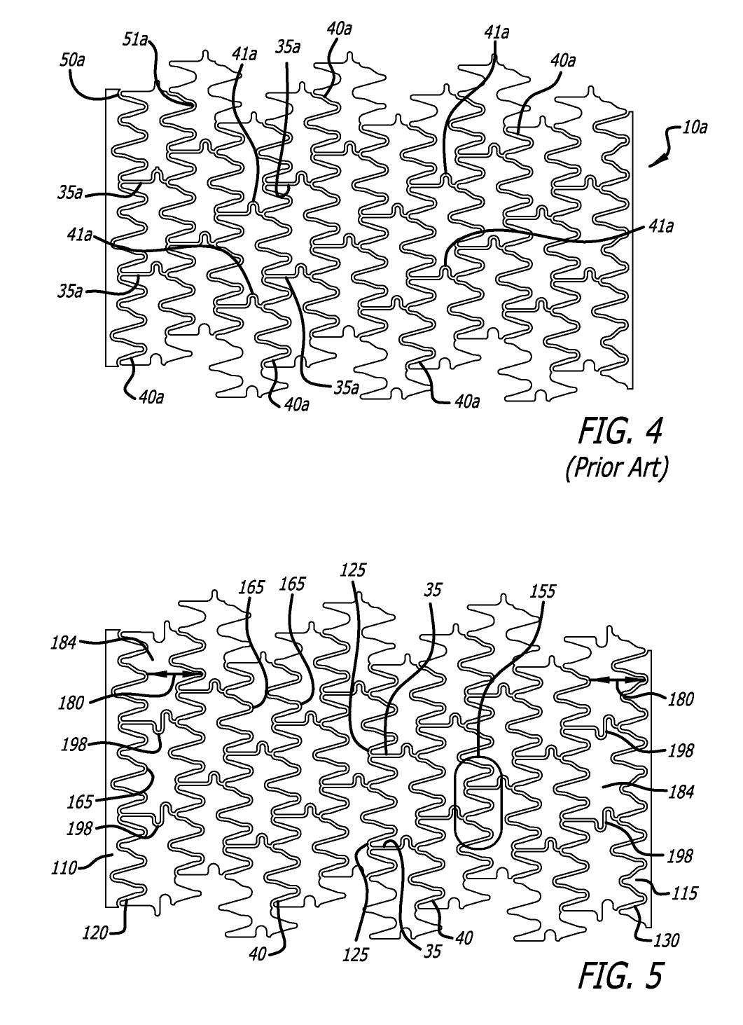

FIG. 4 depicts a prior art stent 10a having a particular undulation pattern and configuration. The stent embodiments and patterns as disclosed herein are illustrative and by way of example only. Other patterns embodying the invention discussed herein can vary and still incorporate the stent retention and flexibility features of the present invention. Referring to FIG. 4, stent 10a is shown in a flattened condition so that the pattern can be clearly viewed, even though the stent is in a cylindrical form in use. The stent is typically formed from a tubular member, however it can be formed from a flat sheet such as shown in FIG. 4 and rolled into a cylindrical configuration as shown in FIG. 2.

The stent 10a is made up of a plurality of cylindrical body rings 40a which extend circumferentially around the stent when it is in a tubular form. Each cylindrical body ring 40a has a cylindrical ring distal end 50a and a cylindrical ring proximal end 51a. Typically, since the stent is laser cut from a tube, there are no discreet parts such as the described cylindrical rings and links. However, it is beneficial for identification and reference to various parts to refer to the cylindrical rings and links and other parts of the stent as separate elements.

Each cylindrical body ring 40a defines a cylindrical plane which is a plane defined by the proximal and distal ends of the ring and the circumferential extent as the cylindrical ring travels around the cylinder. Each cylindrical ring 40a includes a cylindrical outer wall surface which defines the outermost surface of the stent and a cylindrical inner wall surface which defines the innermost surface of the stent. The cylindrical plane follows the cylindrical outer wall surface. The rings 40a shown in FIG. 4 are in phase with adjacent rings, meaning that the peaks in one ring are axially aligned with the peaks in the adjacent rings. Adjacent rings are interconnected by longitudinal links or struts 35a that are substantially straight and substantially aligned with the longitudinal axis of the stent. In the embodiment shown, each strut 35a includes a U-shaped member 41a that flexes in the axial direction, contributing to the overall flexibility of the stent 40a. The longitudinal struts 35a are circumferentially spaced around the stent 10a, such as for example in 30, 60, 90, or 120 degree intervals. In the prior art stent 10a, the spacing in between rings 40a remains constant throughout the stent.

Turning to the stent of the present invention as illustrated by the example in FIG. 5, the stent 10 has at its distal end 110 with a distal end ring 120, and at its proximal end 115 with a proximal end ring 130. The rest of the stent is made up of intermediate rings 40 that are similar to the rings 40a of FIG. 4. Each ring in the embodiment disclosed in FIG. 5 has nine crests or peaks 165 and three links 155. Each ring 40 has W-shaped members 125 that are coupled to the links 35 to connect to the adjacent ring. The rings preferably have a serpentine configuration outside the W-shaped members 125, although other stent configurations are available depending upon the specific application, and the various ring patterns are all included within the scope of the present invention. The rings shown in FIG. 5 are in phase with adjacent rings, meaning the peaks 165 of one ring are aligned in the axial direction with the peaks 165 of the adjacent rings. However, the rings can be out of phase (peak to valley) or some variation thereof without departing from the scope of the present invention.

To improve the stent retention without compromising the radial strength and scaffolding of the stent, the stent is designed with larger spacing between the end rings 120, 130 and their neighboring rings. This spacing, or gap 180, allows more balloon to protrude through the windows 184 between the rings and links 35. This raised balloon protrusion 99 at each end of the crimped stent help to secure the stent 10 on the balloon 22. The gap 180 between the peaks 165 of the distal ring 120 and peaks 165 of the adjacent ring represents a greater spacing between the two adjacent rings as measured in the axial direction along the circumferential plane than between any other two rings except the gap 180 between the proximal ring 130 and the adjacent ring. In one preferred embodiment, the gap 180 can be at least 0.001 inches longer than the other gaps. These gaps 180 create a slightly larger window where additional balloon material can penetrate through the stent 10 as the stent is crimped onto the balloon 22 for anchoring the stent 10 on the balloon 22 to secure the stent during the delivery process. At the gaps 180 at each end of the stent 10, additional balloon material 99 (See FIG. 1a) is forced through the crimped stent. The increase in gap distance 180 allows the elongated struts between the end rings 120, 130 and their neighboring rings to be longer than the interior struts connecting the intermediate rings 40. This increased length of the elongate struts permits one or more additional turns 198 in the flexure portion of the struts 35 to be incorporated in these struts between the end rings 120, 130 and their neighboring rings. The additional turns provide even greater flexure over the single turn or U-shaped struts of FIG. 4, furthering the goal of improved overall flexibility of the stent 10 over the prior art.

The stent 10 of the present invention can be mounted onto a balloon catheter similar to the catheter shown in the prior art device in FIG. 1. The stent is tightly compressed or crimped onto the balloon portion of the catheter and remains tightly crimped on the balloon during delivery through the patient's vascular system. When the balloon is inflated, the stent expands radially outwardly into contact with the body lumen, for example, a renal or coronary artery. When the balloon portion of the catheter is deflated, the catheter system is withdrawn from the patient, and the stent remains implanted in the artery.

The stent 10 of the present invention can be made in many ways. One method of making the stent is to cut it from a thin-walled tubular member, such as stainless steel tubing to remove portions of the tubing in the desired pattern for the stent, leaving relatively untouched the portions of the metallic tubing which are to form the stent. The stent also can be made from other metal alloys such as tantalum, nickel-titanium, cobalt-chromium, titanium, shape memory and superelastic alloys, and noble metals such as gold or platinum. In accordance with the invention, it is preferred to cut the tubing in the desired pattern by means of a machine-controlled laser as is well known in the art.

The stent of the present invention also can be made from shape memory alloys. Shape memory alloys are well known and include, but are not limited to, nickel-titanium and nickel-titanium-vanadium. Any of the shape memory alloys can be formed into a tube and laser cut in order to form the pattern of the stent of the present invention. As is well known, the shape memory alloys can include the type having superelastic or thermoelastic martensitic transformation or display stress-induced martensite. These types of alloys are well known in the art and need not be further described here. If the stent of the present invention is made of a self-expanding metal alloy, such as nickel-titanium or the like, the stent may be compressed or crimped onto a catheter, and a sheath (not shown) is placed over the stent to hold it in place during delivery through the patient's vascular system. Such sheaths are well known in the art. Once the stent has been positioned at the target treatment site, the sheath is withdrawn to deploy the stent. After deployment, the catheter is removed, and the stent remains implanted in the artery.

The present invention stent is ideally suited, for example, for drug delivery (i.e., delivery of a therapeutic agent) since it has a uniform surface area which ensures uniform distribution of drugs. Typically, a polymer is coated onto the stent of the type disclosed in U.S. Pat. Nos. 6,824,559 and 6,783,793 which are incorporated herein by reference. These bioactive agents can be any agent, which is a therapeutic, prophylactic, or diagnostic agent. These agents can have anti-proliferative or anti-inflammmatory properties or can have other properties such as antineoplastic, antiplatelet, anti-coagulant, anti-fibrin, antithrombonic, antimitotic, antibiotic, antiallergic, antioxidant as well as cytostatic agents. Representative embodiments of the active component include actinomycin D (available from Sigma-Aldrich; or Cosmegen® available from Merck) or derivatives, analogs or synonyms thereof, such as dactinomycin, actinomycin IV, actinomycin I.sub.1, actinomycin X.sub.1, and actinomycin C.sub.1; podophyllotoxins such as etoposide and teniposide (Bristol Myers Squibb and Sigma Chemical); cephalotin (Bristol Myers Squibb); trapidil; ticlopidine (Danbury Pharma, Genpharm); tranilast (SmithKline Beecham and LG Chemical Kissei, Japan); IIb-IIIa inhibitors such as eptifibatide (COR therapeutic); clobetasol (Glaxo Wellcome); COX-2 inhibitors such as celecoxib (CELEBREX) (Searle and Pfizer) and rofecoxib (VIOXX) (Merck); PGE1 or alprostadil (Bedford); bleomycin; ENDOSTATIN (EntreMed); ANGIOSTATIN (EntreMed); thalidomide; 2-methoxyestraidol (EntreMed and Sigma Chemical) curcimin (the major constituent of turmeric power extract from the rhizomes of the plant Curcuma longa L found in south and southeast tropical Asia); cisplatin (Sigma Chemical); dipyridamole; tirofiban; verapamil; vitronectine; argatroban; and carboplatin (Sigma Chemical). Additionally corticosteroids such as anti-inflammatory glucocorticoids including clobetasol, diflucortolone, flucinolone, halcinonide, and halobetasol can also be used.

In one embodiment, faster acting non-steroidal anti-inflammatory agents such as naproxen, aspirin, ibuprofen, fenoprofin, indomethacin, and phenylbutazone can be used in conjunction with the glucocorticoids. The use of a non-steroidal anti-inflammatory agent is useful during the early stages of the inflammation in response to a mechanically mediated vascular injury. Examples of suitable therapeutic and prophylactic agents include synthetic inorganic and organic compounds, proteins and peptides, polysaccharides and other sugars, lipids, and DNA and RNA nucleic acid sequences having therapeutic, prophylactic or diagnostic activities. Nucleic acid sequences include genes, antisense molecules which bind to complementary DNA to inhibit transcription, and ribozymes. Some other examples of other bioactive agents include antibodies, receptor ligands, enzymes, adhesion peptides, blood clotting factors, inhibitors or clot dissolving agents such as streptokinase and tissue plasminogen activator, antigens for immunization, hormones and growth factors, oligonucleotides such as antisense oligonucleotides and ribozymes and retroviral vectors for use in gene therapy. Examples of anti-proliferative agents include rapamycin and its functional or structural derivatives, 40-O-(2-hydroxy)ethyl-rapamycin (everolimus), and its functional or structural derivatives, paclitaxel and its functional and structural derivatives. Examples of rapamycin derivatives include methyl rapamycin, ABT-578, 40-O-(3-hydroxy)propyl-rapamycin, 40-O-[2-(2-hydroxy)ethoxy]ethyl-rapamycin, and 40-O-tetrazole-rapamycin. Examples of paclitaxel derivatives include docetaxel. Examples of antineoplastics and/or antimitotics include methotrexate, azathioprine, vincristine, vinblastine, fluorouracil, doxorubicin hydrochloride (e.g. Adriamycin® from Pharmacia & Upjohn, Peapack N.J.), and mitomycin (e.g. Mutamycin® from Bristol-Myers Squibb Co., Stamford, Conn.). Examples of such antiplatelets, anticoagulants, antifibrin, and antithrombins include sodium heparin, low molecular weight heparins, heparinoids, hirudin, argatroban, forskolin, vapiprost, prostacyclin and prostacyclin analogues, dextran, D-phe-pro-arg-chloromethylketone (synthetic antithrombin), dipyridamole, glycoprotein IIb/IIIa platelet membrane receptor antagonist antibody, recombinant hirudin, thrombin inhibitors such as Angiomax a (Biogen, Inc., Cambridge, Mass.), calcium channel blockers (such as nifedipine), colchicine, fibroblast growth factor (FGF) antagonists, fish oil (omega 3-fatty acid), histamine antagonists, lovastatin (an inhibitor of HMG-CoA reductase, a cholesterol lowering drug, brand name Mevacore® from Merck & Co., Inc., Whitehouse Station, N.J.), monoclonal antibodies (such as those specific for Platelet-Derived Growth Factor (PDGF) receptors), nitroprusside, phosphodiesterase inhibitors, prostaglandin inhibitors, suramin, serotonin blockers, steroids, thioprotease inhibitors, triazolopyrimidine (a PDGF antagonist), nitric oxide or nitric oxide donors, super oxide dismutases, super oxide dismutase mimetic, 4-amino-2,2,6,6-tetramethylpiperidine-1-oxyl (4-amino-TEMPO), estradiol, anticancer agents, dietary supplements such as various vitamins, and a combination thereof. Examples of anti-inflammatory agents including steroidal and non-steroidal anti-inflammatory agents include tacrolimus, dexamethasone, clobetasol, combinations thereof. Examples of such cytostatic substance include angiopeptin, angiotensin converting enzyme inhibitors such as captopril (e.g. Capoten® and Capozide® from Bristol-Myers Squibb Co., Stamford, Conn.), cilazapril or lisinopril (e.g. Prinivil® and Prinzide® from Merck & Co., Inc., Whitehouse Station, N.J.). An example of an antiallergic agent is permirolast potassium.

Other therapeutic substances or agents which may be appropriate include alpha-interferon, bioactive RGD, and genetically engineered epithelial cells. The foregoing substances can also be used in the form of prodrugs or co-drugs thereof. The bioactive agents also include metabolites of the foregoing substances and prodrugs of these metabolites. The foregoing substances are listed by way of example and are not meant to be limiting. Other active agents which are currently available or that may be developed in the future are equally applicable.

While particular forms of the invention have been illustrated and described, it will be apparent to those skilled in the art that various modifications can be made without departing from the spirit and scope of the invention. Accordingly, it is not intended that the invention be limited except by the appended claims.

We claim:

1. A stent having a plurality of interconnected sinusoidal rings connected by longitudinal struts to form a cylindrical structure comprising of:
a proximal ring, a plurality of intermediate rings, and a distal ring, each ring defining a cylindrical plane and each ring coupled to an adjacent ring via longitudinal struts, a spacing between adjacent rings defined by a distance in the axial direction between the adjacent rings; and
wherein a spacing between the proximal ring and its adjacent ring and a spacing between the distal ring and its adjacent ring are greater than a spacing of any other two adjacent rings.

2. The stent of claim 1 wherein each ring in the stent are in phase with its adjacent rings, and the spacing between rings is a distance between peaks of said adjacent rings.

3. The stent of claim 1 further comprising elongated struts adjoining the proximal ring with its adjacent ring and elongated struts adjoining the distal ring with its adjacent ring, these elongated struts having a length that is greater than a length of any other longitudinal struts.

4. The stent of claim 3 wherein the longitudinal struts have a linear portion and a flexure portion, and further wherein the elongated struts have a linear portion and a flexure portion, where the flexure portion of the elongated struts permit greater flexure as compared with the longitudinal struts.

5. The stent of claim 4 wherein the flexure portion of the longitudinal struts comprise a U-shaped member, and the flexure portion of the elongated struts have multiple U-shaped members.

6. The stent of claim 5 wherein the flexure portion of the elongated strut comprises two U-shaped members.

7. The stent of claim 1 wherein the spacing between the proximal ring and its adjacent ring and the spacing between the distal ring and its adjacent ring, are at least 0.001 inches greater than a spacing of any other two adjacent rings.
